(19) United States
(12) Patent Application Publication
(10) Pub. No.: US 2022/0096127 A1
(43) Pub. Date: Mar. 31, 2022

(54) PROSTHETIC DEVICE USING HIGH-STRENGTH HYDROGEL

(71) Applicant: Jung-wan SOHN, Seongnam-si (KR)

(72) Inventor: Jung-wan SOHN, Seongnam-si (KR)

(21) Appl. No.: 17/535,843

(22) Filed: Nov. 26, 2021

Related U.S. Application Data
(63) Continuation of application No. PCT/KR2021/001853, filed on Feb. 10, 2021.

(30) Foreign Application Priority Data
Apr. 3, 2020 (KR) 10-2020-0041190

(51) Int. Cl. A61B 17/56; A61L 27/58; A61F 2/00; A61L 27/54; A61L 27/52; A61F 2/30

(52) U.S. Cl. CPC A61B 17/562; A61L 27/58; A61F 2/0077; A61L 27/54; A61L 2430/30; A61L 2430/24; A61B 2017/564; A61B 2017/561; A61F 2002/30754; A61L 27/52

(57) ABSTRACT

The present invention provides a prosthetic device including a pad-shaped spacer (100) including expandable layers (30) having a structure that expands in response to the introduction of moisture because the expandable layers (30) are made of a hydrogel material, so that in a surgery or treatment process, water is absorbed into the expandable layers (30) and fill a space inside a muscle or joint (J) tissue in a state in which the spacer (100) is implanted inside the space inside the muscle or joint (J) tissue until a new tissue grows inside the muscle or joint (J) tissue for a predetermined period of time, and the expandable layers (30) degrade gradually at a rate corresponding to a rate at which the tissue grows, with the result that the muscle or joint (J) tissue can be used during a recovery period.

### CROSS-REFERENCE TO RELATED APPLICATIONS

This application is a continuation of pending PCT International Application No. PCT/KR2021/001853, filed on Feb. 10, 2021, which claims priority from Korean Patent Application No. 10-2020-0041190, filed on Apr. 3, 2020, all of which are incorporated herein by reference in their entirety.

### TECHNICAL FIELD

The present invention relates to a prosthetic device, and more particularly to a prosthetic device that fills the internal space of a muscle or joint until a living tissue grows after the surgery or treatment of the joint or muscle.

### BACKGROUND ART

Conventionally, even when a space such as that in the left photograph of FIG. 1 is generated in a surgical site, surgery is completed in the manner of enclosing a ligament and tissue with a suture, as shown in the right photograph of FIG. 1.

In this case, more specifically, in the case of performing rotator cuff repair as sequentially shown in FIG. 2, surgery is not performed while directly observing the inside of a joint, but an arthroscope is inserted into a portion after cutting the portion and then surgery is performed while viewing a monitor screen.

As a result, the distance between the distal end of a surgical instrument and a hand is long, and it is necessary to indirectly control the surgical instrument through a monitor. Accordingly, unless the person who performs the surgery is a highly skilled surgeon, he or she has to perform long-period surgery for more than an hour after performing general anesthesia.

Furthermore, in order to recover regenerable tissue in a surgical site after surgery and to stabilize a sutured state, it is necessary to steadily perform rehabilitation movement within a set period of time.

Meanwhile, when muscles are not recovered because there is an empty space in a surgical site, the movement for rehabilitation causes considerable pain. Accordingly, a rehabilitation procedure is bound to be a series of tremendous pains.

In order to overcome these problems, conventionally, there has been developed a technology in which a tube-shaped instrument is inserted into a surgical site and the inside of a tube is filled with a liquid through a special nozzle during a surgical procedure, so that the surgical site is protected and so that pain is considerably reduced by minimizing the movement of the surgical site during a rehabilitation procedure, thereby facilitating the rehabilitation procedure, as shown in U.S. Pat. No. 9,770,337 B2 or FIG. 3.

However, in the prior art shown in FIG. 3, since the liquid filled in the tube must not leak, the nozzle for injecting the liquid into the tube needs to be equipped with precision parts having special structures at the end so that a nozzle insertion hole does not remain in the tube after the nozzle is separated from the tube. Furthermore, in order to prevent liquid from leaking, the tube must be prevented from dissolving inside the human body, so that separate removal surgery is required to remove the tube later. Accordingly, there are problems in that a patient's recovery is slow and a lot of cost is incurred.

Therefore, there is a demand for a technology that removes the need for removal surgery to take a tube out later while maintaining the actions of stabilizing a surgical site and reducing pain as in the prior art of FIG. 3, thereby reducing costs and stress.

### DISCLOSURE

### Technical Problem

Accordingly, the present invention is intended to provide a prosthetic device that is inserted into a surgical site in order to fill an empty space in the surgical site or to stabilize the shape of the surgical site, the prosthetic device removing the need for removal surgery to take a tube out later while maintaining the actions of stabilizing of the surgical site and reducing pain and also eliminating costs and stress because there is no need for an airtight structure.

### Technical Solution

In order to accomplish the above object, the present invention provides a prosthetic device including a pad-shaped spacer (100) including expandable layers (30) having a structure that expands in response to the introduction of moisture because the expandable layers (30) are made of a hydrogel material that is a biodegradable polymer and has a fine network structure, so that in a surgery or treatment process, water is absorbed into the expandable layers (30) and fill a space inside a muscle or joint (J) tissue in a state in which the spacer (100) is implanted inside the space inside the muscle or joint (J) tissue until a new tissue grows inside the muscle or joint (J) tissue for a predetermined period of time, and the expandable layers (30) degrade gradually at a rate corresponding to a rate at which the tissue grows, with the result that the muscle or joint (J) tissue can be used during a recovery period, thereby reducing hindrance to activity during the recovery period and also promoting the recovery of the muscle or joint (J) tissue.

In this case, preferably, the spacer (100) further includes shape maintenance layers (20) that are attached to the expandable layers (30).

In this case, preferably, the spacer (100) further includes a core layer (40) having a predetermined rigidity, the expandable layers (30) include two layers and the shape maintenance layers (20) include two layers, and each of the expandable layers (30) and each of the shape maintenance layers (20) are attached to a corresponding side surface of the core layer (40).

In this case, preferably, the expandable layers (30) are attached to both side surfaces of the core layer (40), respectively, the shape maintenance layers (20) are attached to the outer surfaces of the expandable layers (30), respectively, and the two shape maintenance layers (20) attached to the outer surfaces of the expandable layers (30) are connected to each other and form a single bag, so that, even when moisture is absorbed into the expandable layers (30) and thus expansion occurs, an attached state between the shape maintenance layers (20), the expandable layers (30) and the core layer (40) is maintained.

Furthermore, a first pattern (22) configured in a shape of repeating fine protrusions and depressions is formed on a surface of each of the shape maintenance layers (20) attached to a corresponding one of the expandable layers (30), a second pattern (32) configured in a shape corresponding to that of the first pattern (22) is formed on a surface of the expandable layer (30) attached to the shape maintenance layer (20), and the first pattern (22) and the second pattern (32) are attached to each other in such a manner that fine protrusions and fine depressions are engaged with each other, so that, even when expansion occurs in the expandable layers (30), a closely attached state between the shape maintenance layers (20) and the attachment layers is maintained.

In addition, preferably, the prosthetic device according to the present invention further includes a moisture injection unit configured to inject moisture into the spacer (100), and a cell therapy agent for the regeneration of tissue may be uniformly distributed inside the structure of each of the expandable layers (30) or a plurality of capsules in which a cell therapy agent for the regeneration of tissue is contained may be installed to be distributed inside each of the expandable layers (30).

### Advantageous Effects

The prosthetic device according to the present invention is a device that is inserted into a surgical site in order to fill an empty space in the surgical site or to stabilize the shape of the surgical site, and provides the effects of removing the need for removal surgery to take a tube out later while maintaining the actions of stabilizing of the surgical site and reducing pain and also eliminating costs and stress because there is no need for an airtight structure.

### DESCRIPTION OF DRAWINGS

FIG. 1 is a photograph showing an example of a surgical site into which a prosthetic device needs to be inserted;

FIG. 2 is a view sequentially showing a conventional rotator cuff repair procedure;

FIG. 3 is a view showing a prosthetic device according to the prior art;

FIG. 4 is a longitudinal sectional view showing a prosthetic device according to the present invention;

FIG. 5 is a longitudinal sectional view showing a change resulting from the absorption of moisture by the prosthetic device according to the present invention;

FIGS. 6 and 7 are diagrams showing states of use of the prosthetic device according to the present invention;

FIG. 8 is a conceptual view showing an additional embodiment of an expandable layer in the prosthetic device according to the present invention; and

FIG. 9 is an enlarged view showing additional embodiments of the portion indicated by A in FIG. 5.

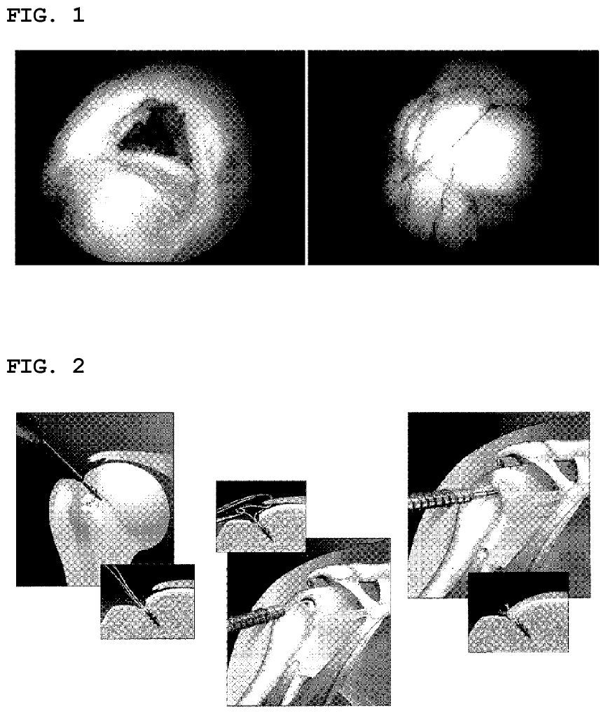

### MODE FOR INVENTION

Specific structural or functional descriptions presented in embodiments of the present invention are merely illustrated as examples for the purpose of describing embodiments based on the concept of the present invention, and the embodiments based on the concept of the present invention may be implemented in various forms. Furthermore, the present invention should not be construed as being limited to the embodiments described herein, and should be understood as including all modifications, equivalents, and substitutes encompassed in the spirit and scope of the present invention.

The present invention will be described in detail below with reference to the accompanying drawings.

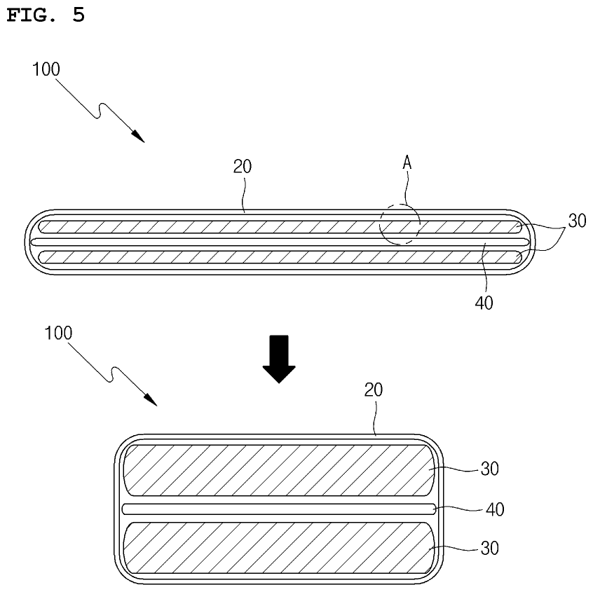

A prosthetic device according to the present invention includes a spacer 100 shown in FIG. 4.

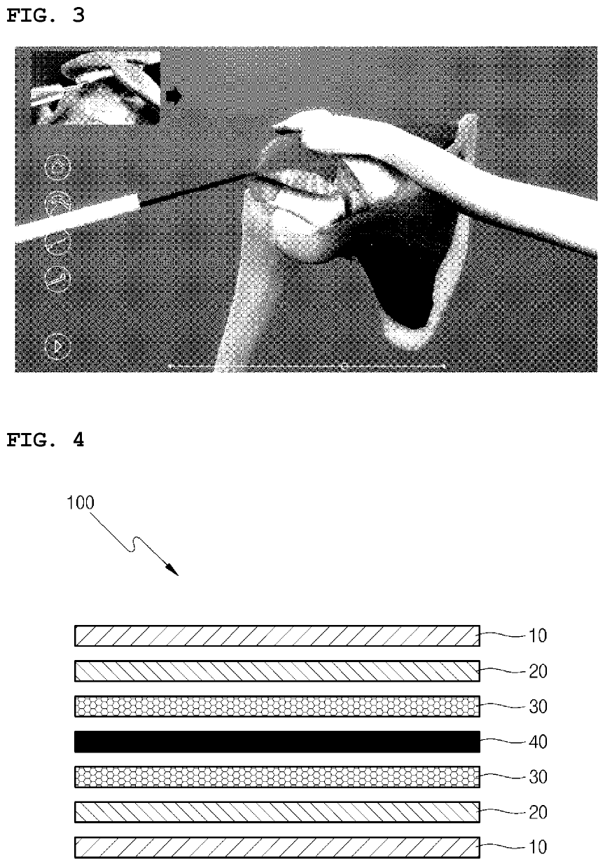

The spacer 100 is inserted into an internal tissue of the human body such as a muscle or joint J in the same manner as the tube, presented in the prior art of FIG. 3 described in the background art section, in the course of surgery or treatment. Accordingly, the spacer 100 stabilizes a surgery or treatment site and allows the muscle or joint J subjected to the surgery or treatment to be used smoothly without pain, thereby promoting recovery while facilitating rehabilitation.

Meanwhile, in the above-described conventional prosthetic device, the principle of filling a space between body tissues corresponds to the principle of inserting a tube into the space and then filling the tube with water, as shown in FIG. 3. Accordingly, the tube in the prior art of FIG. 3 needs to prevent water from leaking in order to maintain pressure. For this purpose, the tip of a nozzle for filling the inside of the tube with water requires complicated and precise parts because a nozzle insertion hole needs to be filled automatically in the process of leaving the tube after filling the tube with water.

Furthermore, according to the prior art of FIG. 3, since it is necessary to prevent the tube from dissolving inside the human body and also prevent water from leaking, the inserted tube needs to be removed at a stage where recovery has progressed to some extent, so that surgery for removing the tube is required.

In the present invention, in order to overcome the problems of these two prior arts, there is adopted a hydrogel member that expands when absorbing moisture.

In the embodiment according to FIG. 4, a hydrogel member constituting each expandable layer 30 is manufactured in the form of a film that expands in the thickness direction. Accordingly, when moisture is injected into the expandable layer 30, it expands in a form in which the thickness thereof increases. In this case, since moisture is permeated into the microstructure of the expandable layer 30, the expanded state of the expandable layer 30 may be maintained even without a separate watertight structure.

In particular, in the present invention, since the expandable layer 30 of the hydrogel material is made of a biodegradable polymer, it may dissolve and degrade in the human body at a rate corresponding to the recovery time of the surgical site, thereby eliminating the need for separate removal surgery.

In this case, the hydrogel material may be formed by crosslinking a biocompatible polymer, selected from hyaluronic acid, hyaluronic acid salt, and a mixture thereof, with a crosslinking agent, or may be formed based on chitosan. Since the hydrogel itself corresponds to a known technology, a further detailed description thereof will be omitted.

The spacer 100 may further include a shape maintenance layer 20 that is attached to the expandable layer 30, as shown in FIG. 4. The shape maintenance layer 20 functions to control the growth of the expandable layer 30 and maintain the stability of the shape. Accordingly, it is preferable that the shape maintenance layer 20 is provided on the outside of the expandable layer 30, so that two shape maintenance layers 20 are provided on respective expandable layers 30. The shape maintenance layers 20 are also made of a biodegradable material so that they can degrade within the human body without separate removal surgery later.

For reference, although the shape maintenance layers 20 may have a thickness of approximately 50 to 200 μm and be manufactured in the form of a film, it may be possible to deviate from these specifications if necessary.

Furthermore, the spacer 100 may further include a core layer 40 having a predetermined rigidity, as shown in FIG. 4. The core layer 40 is a member constituting the overall backbone of the prosthetic device. Accordingly, it has a predetermined rigidity, and may have a predetermined amount of elastic force while allowing plastic deformation because a predetermined amount of deformation needs to be allowed according to the shape of a body part in which the spacer 100 is installed. Since the core layer 40 needs to be also removable without separate surgery, it may be made of a biodegradable polymer.

The core layer 40 may be formed to have a thickness of about 100 to 300 μm or more.

For reference, biomaterials generally used for medical purposes may be classified into bioinert materials that maintain their shape and structure without causing an immune response after transplantation, bioactive materials that directly combine with surrounding tissues and provide biological functions, and biodegradable materials that degrade gradually within the human body, eventually disappear entirely, and are replaced by autologous tissues according to the type of biological reaction with surrounding tissues. In particular, since a biodegradable biomaterial disappears after performing a predetermined function within the living body, there is no need for separate removal surgery and a foreign body reaction, which is a chronic problem of non-degradable biomaterials, may be prevented. Accordingly, all the shape maintenance layers 20 and the core layer 40 constituting parts of the spacer 100 are made of the biodegradable biomaterials.

More specifically, in the structure of the spacer 100, the expandable layers 30 are attached to both side surfaces of the core layer 40, respectively, and the shape maintenance layers 20 are attached to the outer surfaces of the expandable layers 30, i.e., the surfaces of the expandable layers 30 attached to both side surfaces of the core layer 40 and facing the directions opposite to the direction of the core layer 40, respectively, as shown in FIG. 4. Accordingly, the expandable layers 30 and the shape maintenance layers 20 are sequentially attached to both side surfaces of the core layer 40 with the core layer 40 used as the center.

In this case, the two shape maintenance layers 20 attached to the outer surfaces of the expandable layers 30 are connected to each other and form a bag shape, as shown in FIG. 5. Accordingly, even when moisture is absorbed into the expandable layers 30 and thus expansion occurs, the attached state between the shape maintenance layers 20, the expandable layers 30, and the core layer 40 may be maintained. In other words, when the shape maintenance layers 20 are formed in a bag shape as a whole, a phenomenon in which the expandable layers 30 protrude to the side ends or the expandable layers 30 and the shape maintenance layers 20 are off-centered from each other in the lateral direction may be prevented in a process in which the expandable layers 30 absorb moisture and expand.

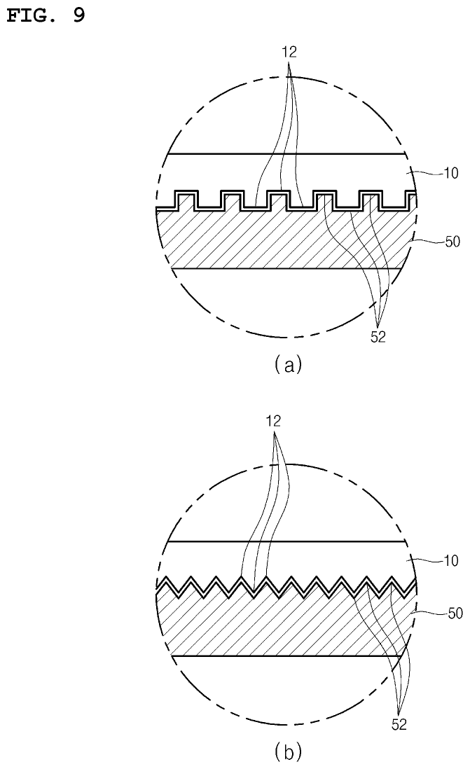

In particular, in order to allow the attachment between the expandable layers 30 and the shape maintenance layers 20 to be maintained even during a process of the expansion of the expandable layers 30, the surfaces of the expandable layers 30 and the shape maintenance layers 20 that are attached to each other may be formed in the structure shown in FIG. 9 so as to maximize frictional force therebetween.

In other words, as shown in FIG. 9, a first pattern 22 configured in the shape of repeating fine protrusions and depressions is formed on the surface of each of the shape maintenance layers 20 attached to a corresponding one of the expandable layers 30, a second pattern 32 configured in a shape corresponding to that of the first pattern 22 is formed on the surface of the expandable layer 30 attached to the shape maintenance layer 20, and the first pattern 22 and the second pattern 32 are attached to each other in such a manner that fine protrusions and fine depressions are engaged with each other. Accordingly, even when expansion occurs in the expandable layers 30, the closely attached state between the shape maintenance layers 20 and the attachment layers may be maintained.

In this case, although the sectional shapes of the first pattern 22 and the second pattern 32 are illustrated as examples in FIGS. 9(a) and 9(b), the first pattern 22 and the second pattern 32 are not necessarily limited thereto. Furthermore, as long as the first pattern 22 and the second pattern 32 are formed in shapes that can be engaged with each other, the shapes of the protrusions and the depressions may vary.

Furthermore, although not shown in the drawings, mutually corresponding patterns similar to the first and second patterns 32 may be formed between the expandable layers 30 and the core layer 40 and increase the attachment force between them.

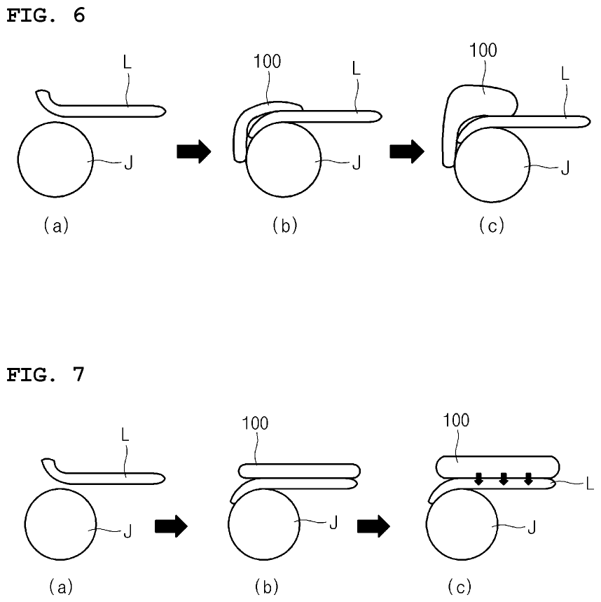

For reference, the forms in which the prosthetic device according to the present invention operates are illustrated as examples in FIGS. 6 and 7. Although conceptually shown here, the spacers 100 shown in FIGS. 6 and 7 expand and act to apply pressure from the top of a ligament L or the top and side of a ligament L so that the ligament L can be operated properly without being separated from a surgically connected point. However, the specific action of the spacer 100 may vary depending on the shape and type of an affected part.

Meanwhile, the prosthetic device according to the present invention may be provided with a moisture injection unit (not shown) configured to inject moisture into the spacer 100. Although the expandable layers 30 absorb moisture from body tissues in the form of a body fluid or other forms, there may occur a situation in which the expandable layers 30 need to rapidly absorb moisture, or there may be a case where moisture injection from the outside is required for other reasons. In order to prepare for such situations, the separate moisture injection unit (not shown) may be provided.

In addition, as described above, the shape maintenance layers 20, the expandable layers 30, and the core layer 40 constituting parts of the spacer 100 are all made of biodegradable materials and the degradation rate of the biodegradable materials may be adjusted according to a mixing ratio in a manufacturing process, so that the spacer may degrade at a rate corresponding to the recovery rate of an affected area into which the spacer is inserted. For this purpose, the spacer 100 may be manufactured in various forms according to the degradation rate, and may be manufactured in various sizes and shapes according to the type of affected part.

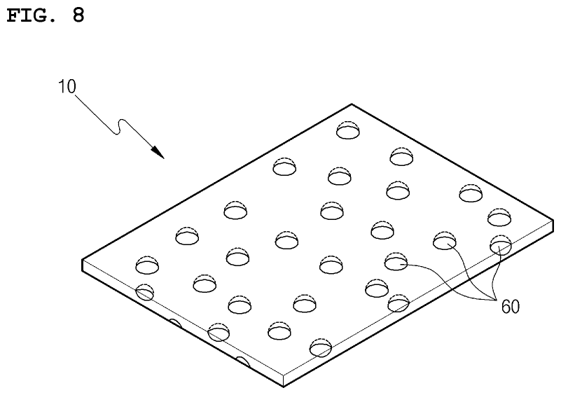

Meanwhile, as shown in FIG. 8, in each of the expandable layers 30, a cell therapy agent for the regeneration of tissue may be uniformly distributed as additives 31 inside the structure of the expandable layer 30, or a plurality of capsules in which a cell therapy agent for the regeneration of tissue is contained may be installed to be distributed inside the expandable layer 30.

In this case, the cell therapy agent is, e.g., a copolymer of PLGA, PLLA, or PGA. The cell therapy agent may control the degradation of a support ideal for the regeneration of tissue because it has the advantage of being able to control the degradation rate according to the copolymerization molar ratio, and may provide high mechanical strength because it can have a hard physical property. PLCL, poly(L-lactide-co-caprolactone) is composed of a copolymer of PLLA, poly(L-lactic acid) and PCL, poly(caprolactone), and has considerably low degradation rate and exhibits high elasticity, unlike PLGA. For these reasons, PLCL, poly(L-lactide-co-caprolactone) is ideal for the regeneration of heart, skin, and vascular tissues, which are tissues that are subjected to continuous mechanical stimulation.

As shown in FIG. 8, the cell therapy agent may continuously release a bone formation-related material for a long period of time when a bone formation-related material such as curcumin is encapsulated as the cell therapy agent.

For reference, components attached to the outermost portions in FIG. 4 are protective films 10. The protective films 10 are provided to protect the spacer 100 until the spacer 100 is used, and are removed before the spacer 100 is inserted into the human body.

### INDUSTRIAL APPLICABILITY

The present invention described above is not limited by the above-described embodiments and the accompanying drawings, and it will be apparent to those of ordinary skill in the art to which the present invention pertains that various substitutions, modifications and changes may be possible within a scope that does not depart from the technical spirit of the present invention.

1. A prosthetic device comprising a pad-shaped spacer (100) comprising expandable layers (30) having a structure that expands by absorbing moisture from an outside because the expandable layers (30) are made of a hydrogel material that is a biodegradable polymer and has a fine network structure, so that in a surgery or treatment process, water is absorbed into the expandable layers (30) and fill a space inside a muscle or joint (J) tissue in a state in which the spacer (100) is implanted inside the space inside the muscle or joint (J) tissue until a new tissue grows inside the muscle or joint (J) tissue for a predetermined period of time, and the expandable layers (30) degrade gradually at a rate corresponding to a rate at which the tissue grows, with a result that the muscle or joint (J) tissue can be used during a recovery period, thereby reducing hindrance to activity during the recovery period and also promoting recovery of the muscle or joint (J) tissue;
wherein the spacer (100) is formed in a multi-layer structure further comprising shape maintenance layers (20) attached to the expandable layers (30), and the shape maintenance layers (20) are attached to outer surfaces of the expandable layers (30), respectively; and
wherein the two shape maintenance layers (20) attached to the outer surfaces of the expandable layers (30) are connected to each other and form a single bag, so that, even when moisture is absorbed into the expandable layers (30) and thus expansion occurs, an attached state between the shape maintenance layers (20) and the expandable layers (30) is maintained.

2. The prosthetic device of claim 1, wherein the spacer (100) further comprises a core layer (40) having a predetermined rigidity, and the expandable layers (30) are attached to both side surfaces of the core layer (40), respectively.

3. The prosthetic device of claim 1, wherein:
a first pattern (22) configured in a shape of repeating fine protrusions and depressions is formed on a surface of each of the shape maintenance layers (20) attached to a corresponding one of the expandable layers (30);
a second pattern (32) configured in a shape corresponding to that of the first pattern (22) is formed on a surface of the expandable layer (30) attached to the shape maintenance layer (20); and
the first pattern (22) and the second pattern (32) are attached to each other in such a manner that fine protrusions and fine depressions are engaged with each other;
so that, even when expansion occurs in the expandable layers (30), a closely attached state between the shape maintenance layers (20) and the attachment layers is maintained.
